(19) United States
(12) Plant Patent Application Publication
(10) Pub. No.: US 2010/0095410 P1
(43) Pub. Date: Apr. 15, 2010

(54) Rose plant named 'Benrave'

(50) ;

(76) Inventor: Frank Benardella, Millstone Township, NJ (US)

Correspondence Address: WENDY R. WHITE, 147 LINEBROOK ROAD, IPSWICH, MA, 01938-2913, US

(21) Appl. No.: 12/287,856

(22) Filed: Oct. 14, 2008

(51) Int. Cl. A01H 5/00

(52) U.S. Cl. PLT/116

(57) ABSTRACT

This new rose plant is of a medium size and upright growing habit. It bears its flowers initially one to a stem. The upper surfaces of the petals noticeably change colors as the flower opens and matures.

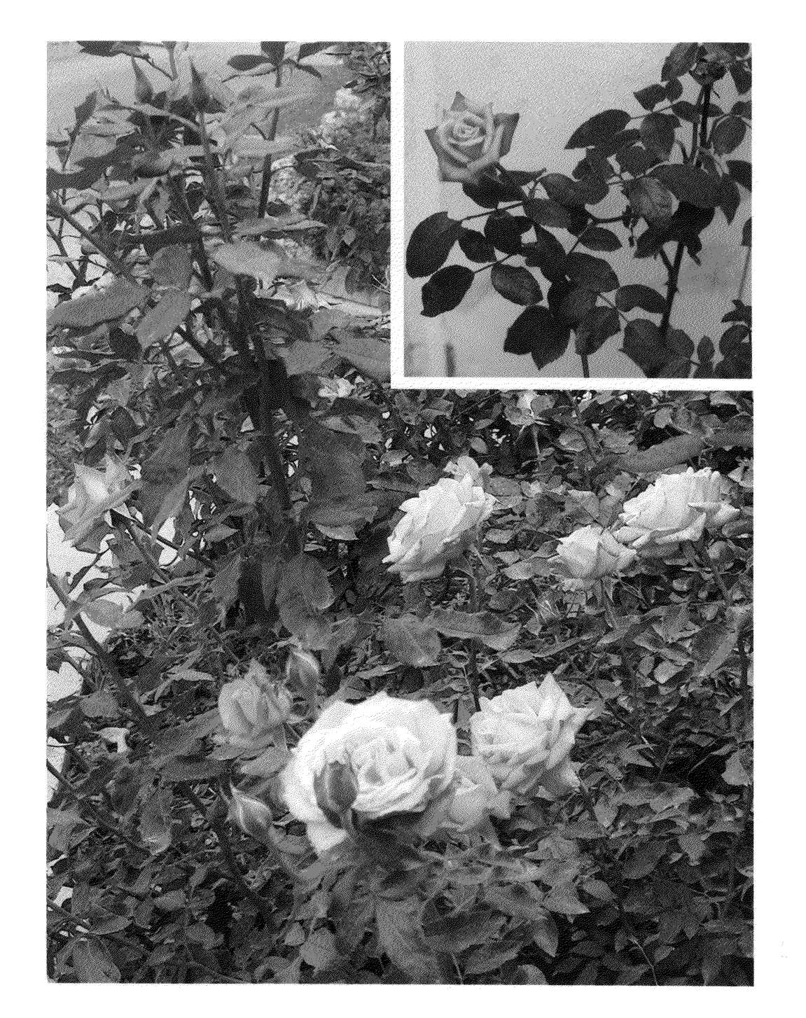

### CROSS REFERENCE

The pollen parent of this new invention is ‘Laguna’, (code name ‘KORmulen’, not patented). A number of plants, related through ‘Laguna’, have been marketed from the same breeding program from which this new rose originated:
Those that are direct descendants of ‘Laguna’ as pollen parent are ‘BENjen’ (U.S. Plant Pat. No. 5,857, expired), a pink-blend, miniature rose; ‘BENblack’ (U.S. Plant Pat. No. 5,925, expired), a dark red miniature rose; ‘BENalav’ (U.S. Plant Pat. No. 6,517), a mauve miniature rose; and ‘BENfig’ (U.S. Plant Pat. No. 8,020), a miniature rose having white flowers edged with pink.
A single rose plant from this breeding that is a direct descendant of ‘Laguana’ as seed parent is a red and white striped, hybrid tea rose named ‘Tinseltown’ (not registered nor patented).
‘The seed parent of this new invention is Jilly Jewel (code name ‘BENmfig’, not patented), a pink blend miniature rose, which is a cross of ‘BENfig’ (see above) by ‘BENmagic’ (U.S. Plant Pat. No. 8,603), a miniature rose having white petals edged with pink. The pollen parent of ‘BENmagic’ is ‘Tinseltown’ (see above). This allows the influence of ‘Laguna’ to be brought in to this new invention through both its pollen parent and seed parent.
   All the above-mentioned roses have hybrid tea form. This new invention begins to open with excellent hybrid tea form, as do the other roses developed in this program, but unlike those roses, when this new invention is grown in direct sunlight it generally has many petaloids displayed in the center of the full open blooms, as is common to ‘Laguna’. This new invention is uniquely colored as it begins to open, with its outer two rows of petals being a deeper and different shade of pink than the inner petals. The flowers and foliage of this new invention are larger than any of these above mentioned roses, except for ‘Tinseltown’. The flowers and foliage of ‘Tinseltown’ are somewhat larger, with flowers averaging four inches in diameter compared to the three inch average diameter of the flowers for this new invention.

### GENUS AND SPECIES

Rosa hybrida

### VARIETAL DENOMINATION

‘BENrave’

### BACKGROUND OF THE INVENTION

The present invention relates to a new and distinct variety of hardy, bush-type rose plant. This new variety is from a single seedling originated by Frank A. Benardella under controlled conditions in a greenhouse in Millstone Township (formerly Englishtown), N.J., by crossing the following two rose plants: The seed parent is a medium pink-blend miniature rose, Jilly Jewel, (code name ‘BENmfig’, not patented). The pollen parent is a red hybrid tea, ‘Laguna’, (code name ‘KORmulen’, not patented).

The primary goal of this breeding program is to produce unique roses with award winning, hybrid tea form on plants having favorable attributes that will increase public appeal. To achieve this goal roses are selected for this hybridizing program primarily for their award winning, hybrid tea form. Pertaining to this particular cross, the pollen parent for this new invention, Laguna, was used because it has been noted to pass on the trait of hybrid tea flower form in a wide range of colors. The seed parent, Jilly Jewel, has been noted to have award winning, hybrid tea form flowers that are usually borne one to a stem. This new invention has the thick, glossy foliage similar to that of Laguna, as well as its upright growing habit. Most of its flowers are borne singly, as those of Jilly Jewel. This resulting new cultivar has been named ‘BENrave’, with the trade name of Focal Point.

### SUMMARY OF THE INVENTION

This present invention relates to a new and distinct variety of a hardy, bush type, rose plant, which has several features that distinguish it from other presently available roses but, primarily, it's unique way of changing its color from bud to full open bloom. Shortly after the sepals open, the color bears resemblance to the red of its pollen parent, Laguna. Half open, it has deep pink outer petals and lighter, muted-pink inner petals. Full open it becomes more of a solid medium pink, near the color of its seed parent, Jilly Jewel. This, in combination with its flower form, upright plant habit, its moderate to strong fragrance, and its dark green, leathery foliage, set this plant apart from all others of which I am aware. Its seed parent, Jilly Jewel, has noticeably smaller foliage, and smaller flowers with an average diameter of two inches and only 17 to 25 petals. This new invention has three inch average diameter flowers that usually have 32 to 56 petals. The flower form and plant habit of the new invention resemble those of its pollen parent, Laguna, but Laguna has larger foliage and orange-red flowers compared to the pink-blend flower on this new invention.

Asexual reproduction of this new cultivar, in Millstone Township, N.J., and Arroyo Grande, Calif. have shown that all distinguishing characteristics of this rose continually come true to form.

### BRIEF DESCRIPTION OF THE DRAWING

The accompanying illustration include a large image showing the foliage, prickles, stems and flowers of this new invention, from bud to full bloom on plants growing in the ground in Arroyo Grande, Calif., as well as a smaller inset image showing two canes, including a single flowering stem, from a plant growing in a container in Ipswich, Mass. These images, taken in September, help to illustrate the differences in color and petal count that may occur under different growing conditions. Color is as nearly correct as it is possible to make in a color illustration of the character.

### BOTANICAL DESCRIPTION OF THE PLANT

The following observations, measurements, values and comparisons are from a five year old plant of Rosa hybrida, ‘BENrave’, grown in a 12 inch plastic nursery container in artificial soil mix, outdoors in Ipswich, Mass., from April to October, 2008, unless otherwise noted. Fertilization was erratic. Phenotypic expression varies with environmental, cultural, and climatic conditions. Color references are made using The Royal Horticultural Society Colour Chart, except where common terms of color are used.

### FLOWERS

BLOOMING HABIT: The new variety bears its flowers, primarily, singly on stems that are slightly crooked at each node. In the summer when the flowers mature, new shoots originate from random nodes below the fading flower, allowing that cane to rebloom every 4 to 5 weeks. In the fall, new side shoots do not develop on flowering canes.

Bud form is ovate with an obtuse base and acute apex. Diameter is usually about 9/16 inch but length may vary from about 7/16 to 12/16 inch.

SEPALS: The flower has five sepals, permanently attached to the receptacle, extending 7/16 inch beyond the tip of the bud just before the sepals divide. Outer surfaces are semi-glossy at the base with the rest of the surface being matte. Scattered across the outer surfaces there is a smattering of stipitate glands and a few glands and hairs, with a slightly heavier concentration at the base of the sepals. The color of the outside surfaces of all sepals is a medium yellow-green, near 144B, and the glands appear near 174A, a dark grayed-orange. Inside surfaces have a fine pubescent covering. The two inner sepals have a heavier concentration of hairs. The inside surface of the three outer sepals appears a light green, near 194A with a triangular basal area of near 194D. The color of the inside surface of the two inner sepals appears a solid grayed-green, near 195B, muted by the pubescence.

The general shape of the individual sepals is lanceolate with acuminate apices. The two outer sepals have foliar appendages along each margin, with the heaviest foliation near the apex. The margins are lined with stipitate glands, including one at the tip of each foliar appendage. Margins of the two inner sepals are heavily ciliated and have no foliar appendages. The one sepal in-between the inner and outer sepals displays characteristics of both the inner and outer sepals, dividing down the center. Sepals unfold ahead of the petals and recurve back to the peduncle, with some crossing over the peduncle and some recurving tightly up to the receptacle. They remain permanently attached to the receptacle and remain in their recurved position.

The widths all five sepals on any individual flower are all the same or nearly the same. On some flowers, the width was measured at or near 5/16 inch, on some ⅛ inch, and on some ⅜ inch. The length of the two outer sepals varies from 1 1/16 to 1⅜ inches. The length of the two innermost sepals ranges from 25/32 to 1⅛ inches. The length of one sepal in-between the inner and outer sepals ranges from 31/32 to 1 1/16 inches. Length of the sepals is not relative to width.

The RECEPTACLE is cuneate, truncated at the top, and the base is tapered to connect evenly to the peduncle. The surface is semi-glossy and pilose. The diameter at its widest point was measured between 11/32 and 16/32 inch and tapered down to between 5/32 and 7/32 inch, respectively, where it attached to the peduncle. The height was measured from 11/32 to 20/32 inch, height was not necessarily respective to diameter. The color is a medium yellow-green, near 144A, occasionally with some flushing on the sunward side of near 173A, from the Greyed-Orange Group. Receptacles remain attached to the peduncle, and if no hip forms, they dry in their attached position.

PEDUNCLES are strong and straight or may be curved slightly sunward. The length of the peduncle varies from 1 15/32 to 2 3/16 inches. Diameters are generally 5/32 inch right below the receptacle, tapering to 3/32 inch before connecting to the flowering stem. The surface of the peduncle is semi-glossy. Some peduncles have a few glands and fewer hairs and gland-tipped hairs, all increasing in quantity toward the base. Others have a few glands and a heavier hispid covering, the entire length of the peduncle. The different vestitures are usually found on the same plant and at the same time. The color is a medium yellow-green, near 144B with heavy flushing near 173A, heaviest right below the receptacle and becoming absent at connection to the stem. Peduncles dry and remain attached to the stem through succeeding bloom-cycles.

BLOOM size averages about 3 inches in diameter, varying from 2 13/16 to 3¼ inches, with a profile depth varying from 1 to 1 13/32 inches, regardless of the diameter but generally being 1¼ inches. Blooms begin to open slowly, high centered, and may take a week or more to go from when the sepals first divide to exhibition stage at half open, which is about 2 to 2½ inches in diameter. Petal counts have been noted to be less on the plants grown in the plastic covered greenhouse or in part shade, having only 29 or 30 petals and 3 to 6 petaloids. Grown in full sun, petal counts were noted to be 32 to 56 with 15 to 24 petaloids. Fragrance is moderate to strong.

PETALS are moderately thick. Both surfaces are satiny. All veins protrude on the upper surface of the outer petals and the main and primary lateral veins also protrude somewhat on the reverse, although this is not noticeable on newly opened flowers. Petals are alternatively attached to the outer edge of the receptacle, but the edges of inner petals often wrap partially around each other. Outer petals of a newly opening flower vary in width from 13/16 to 1 5/16 inches with a length varying from 14/16 to 1 3/16 inches, length is not relative to width. Their shape is suborbicular to oblate with a broadly rounded base, and may be notched along the upper margin but not necessarily right at the apex. Intermediate petals are obtuse and may have an erratic margin, being nearly lobed on one side of some of the petals, similar to the thumb on a mitten. Inner petals are narrow-obovate and may have a slight indentation or an indentation and an acute point at the apex. Outer margins on all petals recurve a bit as they start to unfurl, nearly forming a point at the apex.

COLOR intensity on the flowers is very dependent on the intensity of the light and epigenetic factors while they are forming. In filtered light or partial shade, there is usually a very notable difference between the color of the outer two rows of petals and the inner petals. When grown in nutrient rich soil and full sun, there is less of a color difference, as I will try to describe. The following observations were taken from plants grown in full sun in Ipswich, Mass., except where otherwise noted.

When the sepals first divide, on a well fertilized plant grown in full sun, the color usually first seen is a dark red, near 60A from the Red-Purple Group. When grown under somewhat less than ideal conditions, the color first seen was a Currant Red, near 46A. After a long stretch of cloudy and rainy weather, the color was much less intense, near 48B.

During the first few days, on a well fertilized plant grown in full sun and before the petals unfurl, the color of the upper surface of the outer petals may be near 58B, Rose Red, or between 53D and 58A along the margins, and darker Indian Lake Red, between 58A and 59B, at the apices. Approaching the basal area, the color becomes near 54B, a dark shade of Spinel Red. The basal area is near 5C, a light shade of Sulphur Yellow, and becoming near 154C, a light Chartreuse Yellow, toward the point of attachment. There may be streaks of near 59D anywhere on the petal surface, including in the basal area. The Point of attachment is a medium shade of Dresden Yellow, near 5C. The reverse of the petals is initially near 1D, a light shade of Chartreuse Green, and it is suffused and flushed with near 58A. Margins may be near 58A and they may be varying shades between 59A to 60A at the apices. There are usually areas flushed with other shades of dark red, varying between 60B and 60C. The color on the inner petals is a less intense shade of pink, China Rose, near 58D, suffused with varying shades between 58A and 58B. The basal area is near 4D, a very light Primrose Yellow, and becoming darker, near 5C, toward and at the point of attachment. The reverse is Empire Yellow, near 11D, and suffused lightly with near 58A, near the margins, and then the two colors blend, progressing down the petal. The basal area is near 10D with near 4C at attachment.

When grown under somewhat less than ideal conditions and nutrient levels are lower, which can occur quickly in soilless growing mediums, the color of the flower takes on lavender tones. During the first few days, the upper surface of the outer petals is more apt to be near 57A, a Tyrian Purple, along the upper margin, and becoming a Rose Bengal, between 57B and 57D, as it blends down into the petal to just above the basal area. The basal area and down to the point of attachment is near 154C. The reverse of the petals is initially a very light green-yellow, near 1D and it is suffused and flushed with varying shades between 57C and 57D, heaviest along the margins. The three outermost petals have areas flushed heavily with near 57B, and are near 57A along the margins. The basal area is between 154C and 154D, and sometimes some of the color of the petal goes down into the basal area. The point of attachment is near 2C, which lightens to near 2D in the basal area. The color on the inner petals is a yellowish-white, near 158D, and suffused with a light Rose Bengal, between 57C and 57D. The margins are a medium shade of Rose Bengal, near 57B, with a few areas of 57D. The reverse is a very light Egyptian Buff, near 19D, lightly suffused with near 57D. The basal area is near 2C.

After the sepals have separated it takes up to fourteen days for petals to noticeably begin to unfurl but then they expand quickly to full open. When full open, the outer two rows of petals are a purple-red between 53C and 61C along the margins, and have occasional streaks and flecks of near 53C, a Cardinal Red. The basal area is light yellow, between 4D and 2D. The flushing on the reverse has lightened to a rose-red, between 48D and 58D, though there are some darker areas of near 61A, Ruby Red, and near 61C, a deeper Tyrian Purple. The basal area is near 1D. The intermediate petals are a medium to light pink, between 49D and 68D, and becoming lighter, between 49A and 68C, towards the base of the petals. The point of attachment is near 2D. Progressing a bit further toward the center of the flower, the color of the petals deepens to between 49C and 68D along the outer margins and between 49A and 68C towards the base of the petals. The reverse of these intermediate petals is near 49D, French Rose, with some blushing or streaks of near 61B and near 61 C. The color of the inner petals deepens a bit more only along the outer edges to between 49A and 68B, with a basal area of near 2D. The reverse of these inner petals is near 36D, an Orient Pink, with a basal area of near 4D.

As the bloom ages, the petals become yellow-white, near 158D, and are heavily flushed with somewhat lighter pinks: The edges of the outer two rows of petals are flushed between 68D and 84C. The remainder of the petals are flushed near 62A, Rhodamine Pink, along the outer edges, which becomes near 62B, Phlox Pink, as it progresses down the petal. The basal area of all the petals is a yellowish-white, near 158D and the point of attachment is near 4D. The reverse of the petals is white, near 155D, with flushing from the upper surface showing through.

PETALOIDS have the same color and texture as the inner petals. They are misshapen and their width and shape are quite variable. They may be as half petals and may have part of a sterile anther attached; some are cleft; some have wide white streaks along the main vein; some are borne on filaments. They have been measured up to 11/16 inch wide and ¼ to 1 3/32 inches long.

The general tonality of the flower varies with its stages of bloom. Shortly after the sepals divide, the color bears resemblance to the red of its pollen parent, Laguna. As it begins to open, it appears to be a red blend. Half open it appears to have deep pink outer petals and softer muted-pink inner petals. Full open it appears to be a solid medium pink, more closely resembling the color of its seed parent, Jilly Jewel. The general tonality from a distance is a pink blend.

### REPRODUCTIVE ORGANS

ANDROECIUM: STAMENS are arranged around the outer edge of the receptacle but on occasion they have been observed entwined in the lanate covering on the top of the receptacle. Other times the top of the receptacle has been observed as near glabrous. Stamens ranged in quantity from 40 to 105. When there is a villous covering on the top of the receptacle, there may be enations bearing sterile anthers. The actual FILAMENTS originate around the outer edges of the receptacle, adjacent to the petaloids. They vary in length from ⅛ to 2/8 inch. They appear somewhat translucent with the color at the base near 1C, a Chartreuse Green, and becoming near 47B, a medium red, approaching the anther. The color of the ANTHERS is near 164C, a dull, medium brownish-yellow, and the surrounding pollen sacs are a bit darker, near 164B. Not all anthers appear to be virile or fully formed, having no pollen sacs. Pollen is virile.

GYNOECIUM: PISTILS originate from within the center of the top of the receptacle and vary in quantity from about 20 or less to 40 or more. STYLES are a translucent and very pale yellow, near 158C, at the base and becoming a translucent Indian Lake red, near 59B, below the stigma. Their length varies from 2/16 to 5/16 inch long on each flower. They are thin and arcuate with some being undulate but only in the lower, pale yellow portion of the styles. A few short hairs have been observed on this pale yellow portion. Some styles are fused, two or more together; sometimes they appear as a conflation of a compound pistil; and occasionally they have been observed fused only at the stigma. STIGMAS are near 59D. The stigmas are receptive to pollen.

HIPS have not been observed to maturity. A single hip is now forming but is still green as the receptacle. It has somewhat of an urn shape. The diameter at its widest point is 9/16 inch. The base is oblate, giving one side the height of ½ inch and the other side a height of ⅝ inch. There is a single seed protruding from the top of this hip that is an orange-brown, between 173A and 176A. Stipules are still attached in their recurved position.

### PLANT

The plant of this new rose is upright growing, with good vigor. The height averages 24 to 36 inches and the plant can grow to become 20 to 26 inches wide. Diameter of the main canes was measured at ⅜ inch and the primary laterals as 2/8 inch. Flowering stems generally have a diameter from 5/32 inch at the base tapering to 4/32 inch just below the peduncle and were slightly crooked at each node. The first flowering stems in the spring were measured 7 to 14⅜ inches long to the base of the peduncle, with 9 to 12 nodes per stem, relative to the stem length. When flowers began to fade, new shoots originated from random nodes below the fading flower. These new flowering stems were measured as short as 1⅝ inches and as long as 6 3/16 inches, generally depending how far down on the cane they originated, so that each new flower opens at or above the plant canopy. The plant continues this cycle with each flowering stem. All flowering stems during the summer had 6 to 7 nodes, regardless of the length of the stem.

FOLIAGE: Leaves generally have five leaflets, though some only have three leaflets and some have seven. Mature leaves were measured from 3 9/16 to 5 3/16 inches long. Leaflets are narrow-ovate with an acute apex. Terminal leaflets were measured from 1 15/16 to 2 7/16 inches long and 1 to 1 7/16 inches wide; the length of the leaflets is not relative to their width, and the size of the leaflets is not relative to the size of the leaf. The upper surface is semi-glossy and glabrous, having none to a few random hairs. The main vein is entirely recessed, primary lateral veins are barely recessed, and secondary lateral veins protrude every so slightly. The under surface is matte and leathery. Main veins are entirely protruding and primary lateral veins protrude somewhat. Serration along the margins is a medium, simple serration. Each serrate is tipped with a gland. There also may be a few random hairs along the margins.

The color of the upper surface of new foliage is a medium yellow-green, near 144A, sometimes heavily flushed with a deep red-purple, near 187A, and sometimes only lightly to moderately flushed with the same. The reverse is varying shades between 187B and 187C, Anthocyanin coloring is absent from older foliage, which is a very dark green between 139A and 147A on the upper surface and near 147B on the underside.

PETIOLES vary in length from ½ inch to 1 1/16 inch with calipers varying from 4/64 to 7/64 inch. On young foliage, the ridge and groove of the upper side is heavily flushed between 187A and 187B, though sometimes medium yellow-green, near 144A, can be seen under the flushing. The underside is between 144A and 144B, flushed with between 187A and 187B. On mature foliage the color of the ridge and groove is the same as that of the leaf, between 139A and 147A. There are some glands and stipitate glands along the ridge. There is a light to sparse covering of hairs in the groove, increasing in quantity between the stipules. The under surface is a light yellow-green, between 144D and 145A, and has a few glands or stipitate glands, except between the stipules, where it is glabrous.

The color of the RACHIS and PETIOLULES is the same as that of the petioles, though the flushing on both surfaces of the petiolules is a bit darker. The rachis is most often 1 inch long whether it is a 5-leaflet or 7-leaflet leaf. It has been measured as short as ½ inch and as long as 1⅜ inch. The caliper varies from 4/64 to 5/64 inch. There are stipitate glands along the ridge and a few hairs in the groove, which increase to many at the juncture with the petiolules. The underside of the rachis may have three to six stipitate glands and/or prickles randomly located, and none to three hairs at the junctures with the petiolules. On foliage at the end of the summer, the rachis on the leaves on the flowering stems were mostly found to be glabrous or with only a few stipitate glands near the base. The length of petiolules to the terminal leaflet varies from ½ inch to ¾ inch, with a caliper most often of 1/16 inch. Length of petiolules to the other leaflets varies between 1/32 and 2/32 inch. The petiolules only have a few stipitate glands along one ridge, the lower one, closest to the base of the leaf. There are one to a few hairs in the groove located near the base. The undersurface has a few stipitate glands.

Stipules are paired at the base of the petioles. Their length attached to the petiole varies from 6/16 to 7/16 inch. The tips are angled outward at as little as 20° to as much as 85° from the petiole, most often at 45° . Tips on paired petioles are not always angled out to the same degree. The length of the tips ranges from ⅛ to ⅜ inch. Margins are slightly and loosely rolled back. They are lined with stipitate glands, but also glands and gland-tipped lanceolate enations, giving the margin a toothed appearance.

New wood has a few hairs and glands. Prickles may begin to develop on flowering stems before the flowers form. The surface is glossy. The color is a medium yellow-green, between 144A and 146C. The old wood is less yellow, near 137C. After 1 to 2 years, lenticels begin to form on the older canes. They do not completely cover the canes. Their color is a medium grayed-brown, near 199B.

PRICKLES are quite variable on this new rose variety. Generally, the main stalks seem to have none. At the base of each primary lateral, there were found to be numerous, very short prickles, 1/64 inch long, plus a few up to 5/32 inch long. Above that point, there were usually six or seven prickles randomly located in three inches of stem length but sometimes no prickles in a two inch section of stem. They were 2/32 to 5/32 inch long but a few were also measured at 11/32 inch. On secondary laterals there were found to be ten to fifteen prickles in five inches of stem length, randomly located, but usually at least one just below each node. These prickles were measured from 3/32 to 9/32 inch with the shortest ones being near the base of the cane. On the new growth there may be none, or there may be one just below each node, or there may be a cluster of very short prickles, 1/64 inch long, below each node. By mid-fall, with the exception of the flowering stems, nearly all of the other canes had developed clusters of minute prickles, and sometimes a few that were 1/64 inch long, within the bottom two inches of the base of the canes.

The shape of the prickles is curved and/or angled downward, tapering gradually to a sharp point. The shape of the base is obovate. When young the prickles are a deep red, between 183B and 59A. They darken to a medium-brown, near 177A, before becoming a dark brown, between 177A and 200B, with a base of near 177B. As they become older they become a bit lighter, between 200C and 200D and finally a gray brown, between 197A and 199A. When present on the underside of the rachis on new foliage they appear between 187B and 187C. When present on old foliage, they are often a dull green-yellow, near 160A.

VARIATIONS: A difference in day and night temperatures greater than around 30 degrees will cause the plant to send up long, very fast growing canes. These originate at or near the base of the plant and are often referred to as candelabras because of the large number of blooms originating near the top of a usually long cane. On this new invention, these canes have been observed to extend about ten inches above the top of the crown of the plant and always terminate in a large, loose spray of flowers. As may be seen along the left side of the accompanying image, the color of these canes is usually the same as new foliage, being flushed the entire length with near 187B, until flowers open and mature. They gradually become the same color as the older wood on the plant. The diameter at the base of one of these canes is about ½ inch. Flowering stems originating on the candle have a diameter of 5/32 inch at their base and a length of 5 18/32 to 5 28/32 inches. The largest leaves may be found on the candelabras. The length of mature leaves on these canes was measured from 4 11/16 inches long to 5 5/16 inches long. Terminal leaflets were measured around 1¼ to 1⅝ inches at the widest point, 2 7/16 to 3 1/32 inches in length. The length of the petioles was generally ½ inch, of the rachis was 15/16 inch, and the petiolule to the terminal leaflet was 13/16 inch long.

This new cultivar has been tested hardy in U.S.D.A. zones 5 through 9. It tends to be susceptible to blackspot and in certain locations, to powdery mildew. Rust has not been observed.

1. A new and distinct variety of rose plant is claimed, substantially as described and illustrated herein.
